(19) United States
(12) Patent Application Publication
(10) Pub. No.: US 2025/0134465 A1
(43) Pub. Date: May 1, 2025

(54) OPTOACOUSTIC PROBE WITH PROXIMITY DETECTOR

(71) Applicant: Seno Medical Instruments, Inc., San Antonio, TX (US)

(72) Inventor: Tam Do, San Antonio, TX (US)

(73) Assignee: Seno Medical Instruments, Inc., San Antonio, TX (US)

(21) Appl. No.: 19/010,489

(22) Filed: Jan. 6, 2025

Related U.S. Application Data
(63) Continuation-in-part of application No. 17/661,784, filed on May 3, 2022, now Pat. No. 12,186,099.
Application No. 17/661,784 is a continuation of application No. 16/517,831, filed on Jul. 22, 2019, now Pat. No. 11,382,565.

(51) Int. Cl. A61B 5/00; A61B 5/103; G01N 29/24

(52) U.S. Cl. CPC A61B 5/6844; A61B 5/0095; A61B 5/1032; A61B 5/443; G01N 29/2418

(57) ABSTRACT

An optoacoustic probe system with an optoacoustic probe that can include a light source configured to generate light that is transmitted along a light path to generate optoacoustic return signals when the light reacts with the volume and an optical window configured to carry the light along the light path to the volume. The optoacoustic probe can include an auxiliary light source configured to generate auxiliary light and a secondary light sensor configured to detect signals generated from the auxiliary light source reflecting from the volume. The optoacoustic probe system may also have a microcontroller including one or more processors configured to receive the signals generated from the auxiliary light reflecting from the volume, determine contact between the volume and the optoacoustic probe, and prevent the light from the light source emitting from the probe housing until the optoacoustic probe is contacting the volume.

### CROSS-REFERENCE

This application is a Continuation-in-part of an claims benefit to U.S. patent application Ser. No. 17/661,784 filed May 3, 2022, titled “OPTOACOUSTIC PROBE” that was a Continuation of and claimed benefit to U.S. patent application Ser. No. 16/517,831 filed Jul. 22, 2019, titled “OPTOACOUSTIC PROBE”, the subject matter of each of which are herein incorporated by reference in its entirety.

### TECHNICAL FIELD

The present invention relates in general to the field of medical imaging, and in particular to a system relating to optoacoustic imaging.

### BACKGROUND

Optoacoustic imaging systems visualize thin tissue slices noninvasively through skin at a tissue site. A tissue site may contain a variety of tissue structures that may include, for example, tumors, blood vessels, tissue layers, and components of blood. In optoacoustic imaging systems, light is used to deliver optical energy to a planer slice of the tissue site, which as a result of optical absorption with the tissue structures, produces acoustic waves. An image spatially representing the tissue site can be generated by performing image reconstruction on acoustic signals that return to an ultrasound transducer array. Because biological tissue scatters impinging optical energy in many directions the optical energy can be absorbed by tissue structures outside of a targeted region, which can generate acoustic return signals that interfere with the imaging of tissue structures within the targeted region.

A laser light source typically provides the optical energy required to generate the acoustic waves. During such operation, great care must be taken to ensure the emitted optical energy does not harm a patient and clinician, including the eyes of the patient or clinician.

In order to prevent such harm, safety glasses are often worn by the clinician during such procedures. Additionally, probe holders can be utilized that absorb or prevent the emission of the radiation, so that if a clinician forgets to turn off the probe, the radiation cannot harm any individuals in the environment. In other examples, the probe does not emit optical energy unless a foot actuator is utilized. Still, when the probe is removed from the holder, or when a clinical drops the probe while utilizing the foot actuator, potentially harmful optical energy may be emitted into the environment, increasing the chances of harm.

To this end, some optoacoustic probes use contact and/or proximity sensors that are used to detect optoacoustic probe contact prior to emission of light from the optoacoustic probe. For example, a secondary light source that emits radiation that is not harmful to the patient or clinician can be provided where the reflection of the light off the patient can be detected by the optoacoustic probe. Still, problems exist for such systems because of difficulties in capturing the reflected light and analyzing such reflected light can result in errors. For example, not every patient has the same skin color or tone, resulting in inconsistencies in determining the exact location of the optoacoustic probe as a result of this light altering variable.

A need therefore exists for a more effective design to improve optoacoustic safety when determining whether the optoacoustic probe is contacting a patient.

### BRIEF SUMMARY

New and useful systems, apparatuses, and methods for providing optoacoustic imaging are set forth in the appended claims. Illustrative embodiments are also provided to enable a person skilled in the art to make use of the claimed subject matter.

Objectives, advantages, and a preferred mode of making and using the claimed subject matter may be understood best by reference to the accompanying drawings in conjunction with the following detailed description of illustrative embodiments.

In accordance with embodiments herein, an optoacoustic probe for optoacoustic imaging of a volume can be provided that can have a distal end operable to contact the volume and a proximal end. The optoacoustic probe can include a light source outside a probe housing configured to generate light that is transmitted along a light path to generate optoacoustic return signals when the light reacts with the volume and an optical window configured to carry the light along the light path to the volume. The optoacoustic probe can also include an auxiliary light source coupled to the probe housing and configured to generate auxiliary light carried through the optical window to the volume during operation of the optoacoustic probe and a secondary light sensor configured to detect signals generated from the auxiliary light source reflecting from the volume. The optoacoustic probe system may also have a microcontroller including one or more processors, and a memory coupled to the one or more processors, wherein the memory stores program instructions. The program instructions can be executable by the one or more processors to receive the signals generated from the auxiliary light reflecting from the volume, determine contact between the volume and the optoacoustic probe based on the signals generated from the auxiliary light reflecting from the volume and color of a surface of the volume, and prevent the light from the light source emitting from the probe housing until the optoacoustic probe is contacting the volume.

Optionally, the one or more processors can include program instructions to vary an output of the auxiliary light source based on the color of the surface of the volume. In one aspect, the auxiliary light source can be a first auxiliary light source configured to emit a first wavelength of light, and the optoacoustic probe further comprises a second auxiliary light source configured to emit a second wavelength of light. In another aspect, the first wavelength of light and the second wavelength of light can be different wavelengths. In one example, the one or more processors can include program instructions to select between the first auxiliary light source and the second auxiliary light source based on the color of the surface of the volume. In another example, the one or more processors may include program instructions operate the first auxiliary light source and second auxiliary light source to mix the first wavelength of light with the second wavelength of light based on the color of the surface of volume.

Optionally, the light source can be a laser light source and the auxiliary light source is a light emitting diode. In one aspect, the secondary light sensor can be a photodiode. In another aspect, the optoacoustic probe system can also include a triggering system configured to actuate the light source and the auxiliary light source and configured to prevent actuation of the light source before actuation of the auxiliary light source. In one example, the auxiliary light source can be disposed adjacent to the optical window at the distal end of the optoacoustic probe. In another example, the one or more processors can be configured to receive the signals generated from the auxiliary light reflecting from the volume while the light source generates the light.

In accordance with embodiments herein, a method of triggering a light source of an optoacoustic probe is provided. The method can include selecting an auxiliary light source from plural auxiliary light sources based on a color of a surface of a volume, operating the auxiliary light source selected to generate an auxiliary light carried through an optical window to the volume during operation of the optoacoustic probe, and detecting the auxiliary light after the auxiliary light has reflected from the surface of the volume. The method can also include determining when the optoacoustic probe contacts the volume based on the auxiliary light after the auxiliary light has reflected from the volume, triggering the light source in response to determining the optoacoustic probe contacts the volume, and preventing the light source from actuating in response to determining the optoacoustic probe is not contacting the volume.

Optionally, the method can also include varying an output of the auxiliary light source based on the color of the surface of the volume. In one aspect, selecting the auxiliary light source from the plural auxiliary light sources may include identifying a first auxiliary light source configured to emit a first wavelength of light, identifying a second auxiliary light source configured to emit a second wavelength of light, and selecting the first auxiliary light source as the auxiliary light source based on the color of the surface of the volume. In another aspect, the auxiliary light source can be a first auxiliary light source, and the method can also include mixing a first wavelength of light emitted by the first auxiliary light source with a second wavelength of light emitted by a second auxiliary light source based on the color of the surface of the volume. In one example, the method can also include varying an intensity of the auxiliary light source based on the color of the surface of the volume. In another example, the method can also include preventing actuation of the light source before actuation of the auxiliary light source.

In accordance with embodiments herein, an optoacoustic system for optoacoustic imaging of a volume is provided that can include an optoacoustic probe having a distal end operable to contact a surface of the volume and a laser light source in a chassis coupled to the optoacoustic probe via an optoacoustic cable, the laser light source configured to generate light that is transmitted along a light path within the optoacoustic cable to generate optoacoustic return signals when the light reacts with the volume. The optoacoustic system can also include an optical window configured to carry the light along the light path to the volume, a first auxiliary light source positioned adjacent to the optical window at the distal end of the optoacoustic probe and configured to emit a first wavelength of light, and a second auxiliary light source posited adjacent to the optical window at the distal end of the optoacoustic probe and configured to emit a second wavelength of light. The optoacoustic probe system can also include a microcontroller having one or more processors, and a memory coupled to the one or more processors, wherein the memory stores program instructions. The program instructions can be executable by the one or more processors to select at least one of the first auxiliary light source and the second auxiliary light source based on a color of the surface of the volume, operate auxiliary light source selected, and prevent the light from the laser light source from emitting based on signals generated from the auxiliary light source selected.

Optionally, the optoacoustic probe system can include secondary light sensors configured to receive the signals generated from the auxiliary light source selected. In one aspect, the one or more processors may include program instructions to vary an output of the auxiliary light source selected based on the color of the surface of the volume.

### BRIEF DESCRIPTION OF THE DRAWINGS

The foregoing and other objects, features, and advantages of the invention will be apparent from the following more particular description of preferred embodiments as illustrated in the accompanying drawings, in which reference characters refer to the same parts throughout the various views. The drawings are not necessarily to scale, emphasis instead being placed upon illustrating principles of the invention.

FIG. 1 shows a schematic block diagram illustrating an embodiment of an optoacoustic system that may be used as a platform for the methods and devices disclosed herein.

FIG. 2A shows a schematic orthogonal view of an embodiment of a probe that may be used in connection with the methods and other devices disclosed herein.

FIG. 2B shows an end view of an optoacoustic probe that may be used in connection with the methods and other devices disclosed herein.

FIG. 2C shows side view of an optoacoustic probe contacting a surface of a volume in connection with the methods and other devices disclosed herein.

FIG. 2D shows side view of an optoacoustic probe spaced from a surface of a volume in connection with the methods and other devices disclosed herein.

FIG. 3 shows an exploded view of an embodiment of the probe shown in FIG. 2A.

FIG. 4 shows a cross-sectional view of the probe shown in FIG. 3.

FIG. 5 shows a schematic block diagram of a control system for devices disclosed herein.

FIG. 6 shows a schematic diagram of light control circuitry for an optoacoustic probe as disclosed herein.

FIG. 7 shows a block flow diagram of a method of controlling light sources of an optoacoustic probe as disclosed herein.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The following description and drawings are illustrative and are not to be construed as limiting. Numerous specific details are described to provide a thorough understanding. However, in certain instances, well-known or conventional details are not described in order to avoid obscuring the description. References to one or an embodiment in the present disclosure are not necessarily references to the same embodiment; and such references mean at least one.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments, but not other embodiments.

The systems and methods are described below with reference to, among other things, block diagrams, operational illustrations and algorithms of methods and devices to provide optoacoustic imaging with out-of-plane artifact suppression. It is understood that each block of the block diagrams, operational illustrations and algorithms and combinations of blocks in the block diagrams, operational illustrations and algorithms, can be implemented by means of analog or digital hardware and computer program instructions.

These computer program instructions can be stored on computer-readable media and provided to a processor of a general-purpose computer, special purpose computer, ASIC, or other programmable data processing apparatus, such that the instructions, which execute via the processor of the computer or other programmable data processing apparatus, implements the functions/acts specified in the block diagrams, operational block or blocks and or algorithms.

In some cases, frequency domain-based algorithms require zero or symmetric padding for performance. This padding is not essential to describe the embodiment of the algorithm, so it is sometimes omitted from the description of the processing steps. In some cases, where padded is disclosed in the steps, the algorithm may still be carried out without the padding. In some cases, padding is essential, however, and cannot be removed without corrupting the data.

In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

Reference will now be made in more detail to various embodiments of the present invention, examples of which are illustrated in the accompanying figures. As will be apparent to one of skill in the art, the data structures and processing steps described herein may be implemented in a variety of other ways without departing from the spirit of the disclosure and scope of the invention herein and should not be construed as being limited to the embodiments set forth herein. Rather, these embodiments are provided so that this disclosure will be thorough and complete, and will fully convey the concept of the disclosure to those skilled in the art.

Embodiments herein may be implemented in connection with one or more of the systems and methods described in one or more of the following patents, publications and/or published applications, all of which are expressly incorporated herein by reference in their entireties:

U.S. Pat. No. 7,999,161, titled “Laser-Activated Nanothermolysis Of Cells” filed Jul. 23, 2007;

U.S. Pat. No. 9,289,191, titled “System and method for Acquiring Optoacoustic Data and Producing Parametric Maps Thereof”, and filed Jun. 13, 2012;

U.S. Pat. No. 9,517,055, titled “System And Method For Acquiring Optoacoustic Data And Producing Parametric Maps Using Subband Acoustic Compensation” filed Nov. 25, 2013;

U.S. Pat. No. 9,724,072, titled “System And Method For Mixed Modality Acoustic Sampling” filed Dec. 13, 2013;

U.S. Pat. No. 9,456,805, titled “System And Method For Acquiring Optoacoustic Data And Producing Parametric Maps Using Interframe Persistent Artifact Removal” filed Dec. 19, 2013;

U.S. Publication 2016/0199037, titled “System And Method For Acquiring Optoacoustic Data And Producing Parametric Maps thereof” filed Mar. 22, 2016;

U.S. Publication 2017/0035388, titled “System And Method For Mixed Modality Acoustic Sampling” filed Oct. 18, 2016;

U.S. Pat. No. 9,792,686, titled “System And Method For Acquiring Optoacoustic Data And Producing Parametric Maps Using Subband Acoustic Compensation” filed Nov. 17, 2016;

U.S. Publication 2017/0296151, titled “System And Method For Mixed Modality Acoustic Sampling” filed Jun. 30, 2017;

U.S. Publication 2013/0109950, titled “Handheld Optoacoustic Probe” filed Nov. 2, 2011;

U.S. Publication 2016/0296121, titled “Handheld Optoacoustic Probe” filed May 2, 2016;

U.S. Pat. No. 8,686,335, titled “System And Method For Adjusting The Light Output Of An Optoacoustic Imaging System” filed Dec. 31, 2011;

U.S. Pat. No. 9,528,936, titled “System And Method For Adjusting The Light Output Of An Optoacoustic Imaging System” filed Mar. 31, 2014;

U.S. Publication 2017/0108429, titled “System And Method For Adjusting The Light Output Of An Optoacoustic Imaging System” filed Dec. 27, 2016;

U.S. Pat. No. 9,330,452, titled “Statistical Mapping In An Optoacoustic Imaging System” filed Mar. 11, 2013;

U.S. Pat. No. 9,836,838, titled “Statistical Mapping In An Optoacoustic Imaging System” filed May 3, 2016;

U.S. Publication 2018/0061050, titled “Statistical Mapping In An Optoacoustic Imaging System” filed Nov. 6, 2017;

U.S. Pat. No. 9,610,043, titled “System And Method For Producing Parametric Maps Of Optoacoustic Data” filed Jun. 13, 2012;

U.S. Publication 2017/0100040, titled “System And Method For Producing Parametric Maps Of Optoacoustic Data” filed Dec. 21, 2016;

U.S. Publication 2013/0338501, titled “System And Method For Storing Data Associated With The Operation Of A Dual Modality Optoacoustic/Ultrasound System” filed Jun. 13, 2012;

U.S. Publication 2013/0338475, titled “Optoacoustic Imaging System With Fiber Optic Cable” filed Jun. 13, 2012;

U.S. Publication 2014/0194723, titled “Multi-Layer Coating For Optoacoustic Probe” filed Jan. 13, 2014;

U.S. Publication 2017/0150890, titled “Optoacoustic Probe With Multi-Layer Coating” filed Jan. 31, 2017;

U.S. Pat. No. 9,615,750, titled “Methods And Compositions For Carrier Agents And Clearing Agents Used In Optoacoustic Imaging Systems” filed Jun. 14, 2012;

U.S. Publication 2013/0116538, titled “Optoacoustic Imaging Systems And Methods With Enhanced Safety” filed Oct. 19, 2012;

U.S. Publication 2015/0297090, titled “Optoacoustic Imaging Systems And Methods With Enhanced Safety” filed Jan. 23, 2015;

U.S. Publication 2013/0289381, titled “Dual Modality Imaging System For Coregistered Functional And Anatomical Mapping” filed Nov. 2, 2012;

U.S. Pat. No. 9,757,092, titled “Method For Dual Modality Optoacoustic Imaging” filed Nov. 2, 2012;

U.S. Publication 2014/0039293, titled “Optoacoustic Imaging System Having Handheld Probe Utilizing Optically Reflective Material” filed Jan. 22, 2013;

U.S. Publication 2017/0014101, titled “Dual Modality Imaging System For Coregistered Functional And Anatomical Mapping” filed Sep. 27, 2016;

U.S. Publication 2013/0303875, titled “System And Method For Dynamically Varying The Angle Of Light Transmission In An Optoacoustic Imaging System” filed Nov. 2, 2012;

U.S. Pat. No. 9,445,785, titled “System And Method For Normalizing Range In An Optoacoustic Imaging System” filed Dec. 21, 2012;

U.S. Pat. No. 9,282,899, titled “System And Method For Detecting Anomalous Channel In An Optoacoustic Imaging System” filed Dec. 21, 2012;

U.S. Publication 2014/0005544, titled “System And Method For Providing Selective Channel Sensitivity In An Optoacoustic Imaging System” filed Dec. 21, 2012;

U.S. Publication 2016/0317034, titled “System And Method For Providing Selective Channel Sensitivity In An Optoacoustic Imaging System” filed Jul. 11, 2016;

U.S. Pat. No. 9,445,786, titled “Interframe Energy Normalization In An Optoacoustic Imaging System” filed Jan. 22, 2013;

U.S. Publication 2017/0000354, titled “Interframe Energy Normalization In An Optoacoustic Imaging System” filed Sep. 19, 2016;

U.S. Publication 2014/0206978, titled “Probe With Optoacoustic Isolator” filed Jan. 22, 2013;

U.S. Pat. No. 9,743,839, titled “Playback Mode In An Optoacoustic Imaging System” filed Mar. 15, 2013;

U.S. Publication 2017/0332916, titled “Playback Mode In An Optoacoustic Imaging System” filed Jul. 27, 2017;

U.S. Pat. No. 9,398,893, titled “System And Method For Diagnostic Vector Classification Support” filed Mar. 11, 2014;

U.S. Pat. No. 10,026,170, titled “System And Method For Diagnostic Vector Classification Support” filed Jul. 19, 2016;

U.S. application Ser. No. 16/022,138, titled “System And Method For Diagnostic Vector Classification Support” filed Jun. 28, 2018;

U.S. Pat. No. 9,730,587, titled “Diagnostic Simulator” filed Mar. 15, 2013;

U.S. Publication 2017/0332915, titled “Diagnostic Simulator” filed Jul. 27, 2017;

U.S. Pat. No. 8,823,928, titled “Light Output Calibration In An Optoacoustic System” filed Mar. 15, 2013;

U.S. Pat. No. 9,163,980, titled “Light Output Calibration In An Optoacoustic System” filed Jul. 11, 2014;

U.S. Pat. No. 9,814,394, titled “Noise Suppression In An Optoacoustic System” filed Mar. 15, 2013;

U.S. Publication 2018/0078144, titled “Noise Suppression In An Optoacoustic System” filed Nov. 13, 2017;

U.S. Pat. No. 9,733,119, titled “Optoacoustic Component Utilization Tracking” filed Mar. 15, 2013;

U.S. Publication 2017/0322071, titled “Optoacoustic Component Utilization Tracking” filed Jul. 27, 2017;

U.S. Publication 2015/0101411, titled “Systems And Methods For Component Separation In Medical Imaging” filed Oct. 13, 2014;

U.S. Publication 2015/0305628, titled “Probe Adapted To Control Blood Flow Through Vessels During Imaging And Method Of Use Of Same” filed Feb. 27, 2015;

U.S. Publication 2016/0187481, titled “Opto-Acoustic Imaging System With Detection Of Relative Orientation Of Light Source And Acoustic Receiver Using Acoustic Waves” filed Oct. 30, 2015.

Provided is an optoacoustic probe system that includes an optoacoustic probe that has plural auxiliary light sources used to determine when the optoacoustic probe contacts the surface of a volume being scanned to determine when laser light sources can emit radiation. The plural auxiliary light sources can each emit different wavelengths of light or similar wavelengths of light. Based on the color of the surface of the volume to be scanned (e.g., based on the skin color of a patient) a controller can vary the operation of the plural auxiliary light sources to provide a desired light output from the combined plural auxiliary light sources. In example embodiments the optoacoustic probe can be tuned for the different colors of the surface of the volume by using a phantom (e.g., volume formed to mimic the characteristics of a human) and obtaining reflected signals of the auxiliary light sources with secondary light sensors. Based on the feedback, characteristics of light output by a combination of the auxiliary light sources may be varied. In example embodiments the characteristics of the light output varied can include the intensity of light, wavelength of light, or the like. In practice, based on the color of the surface of the volume to be scanned the auxiliary light sources to be operated are selected, and then operation can be adjusted to provide the most reflective light output from plural auxiliary light sources. As a result, accuracy of readings of when the optoacoustic probe contacts a patient's skin increases, enhancing safety of the optoacoustic probe system.

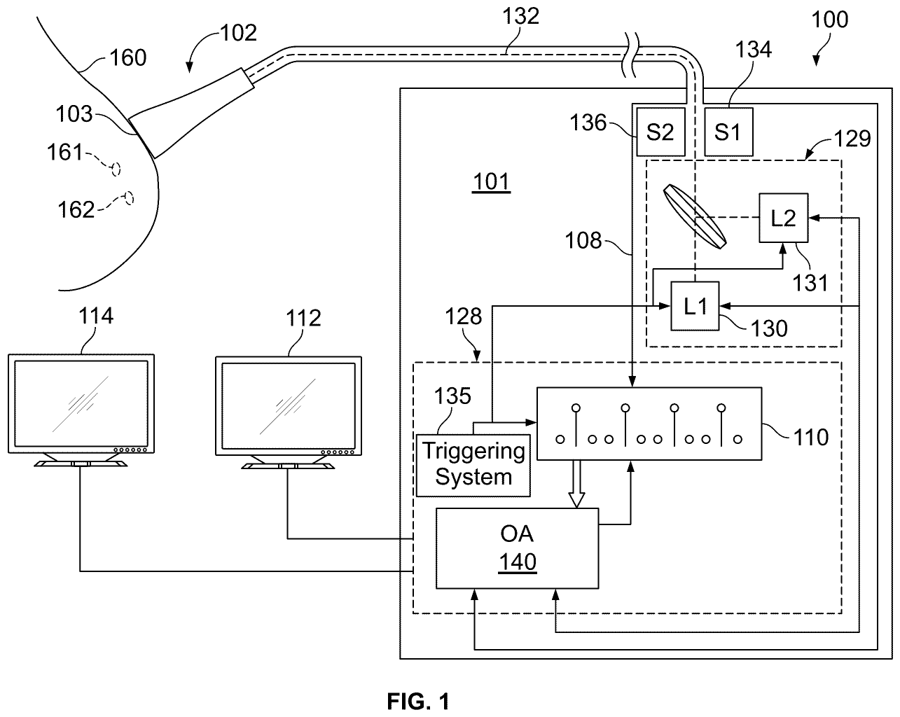

Turning to FIG. 1 device 100 provides an optoacoustic probe system. In an embodiment, the optoacoustic probe system 100 includes an optoacoustic probe 102 connected via a light path 132 and an electrical path 108 to a system chassis 101. Within the system chassis 101 is housed a light subsystem 129 and a computing subsystem 128. The computing subsystem 128 includes one or more computing components for optoacoustic control and analysis; these components may be separate, or integrated. In an embodiment, the computing subsystem comprises an ultrasound system 110, a triggering system 135, an optoacoustic processing and overlay system 140.

In an embodiment, the light subsystem 129 is capable of producing pulses of light of at least two different wavelengths. In an embodiment, the light subsystem 129 includes two separate light sources 130, 131. In one example at least one (or both) of the light sources 130, 131 are laser light sources. In an embodiment the light sources 130, 131 area Nd:YAG laser and an Alexandrite laser. The output of the primary light sources 130, 131 of the light subsystem 129 is delivered to the probe 102 via the light path 132.

The computing subsystem 128 can receive signals or readings from each of the first energy output sensor 134 and the second energy output sensor 136 to identify or determine the energy output of one or more of the light sources 130, 131. In one example the computing subsystem can include a subsystem for determining the confidence level of at least one, if not both of the first energy output sensor 134 and the second energy output sensor 136. In an example, the computing subsystem can be configured to use the energy output identified or determined to dynamically adjust the voltage provided for at least one light source 130, 131 to keep the energy output of the light source 130, 131 between 80%-120% of a target energy output. In an example the computing subsystem determines the expected energy output based on the voltage provided and compares the detected energy output and based on this comparison adjusts the voltage to ensure the energy output remains within the 80%-120% range. So, if the voltage is set to achieve a 100% energy output and one or more of the first energy output sensor 134 and/or second energy output sensor 136 are used to determine the detected energy output is only 70% of the expected energy output, the computing subsystem 128 may cause the output voltage to increase the detected energy output.

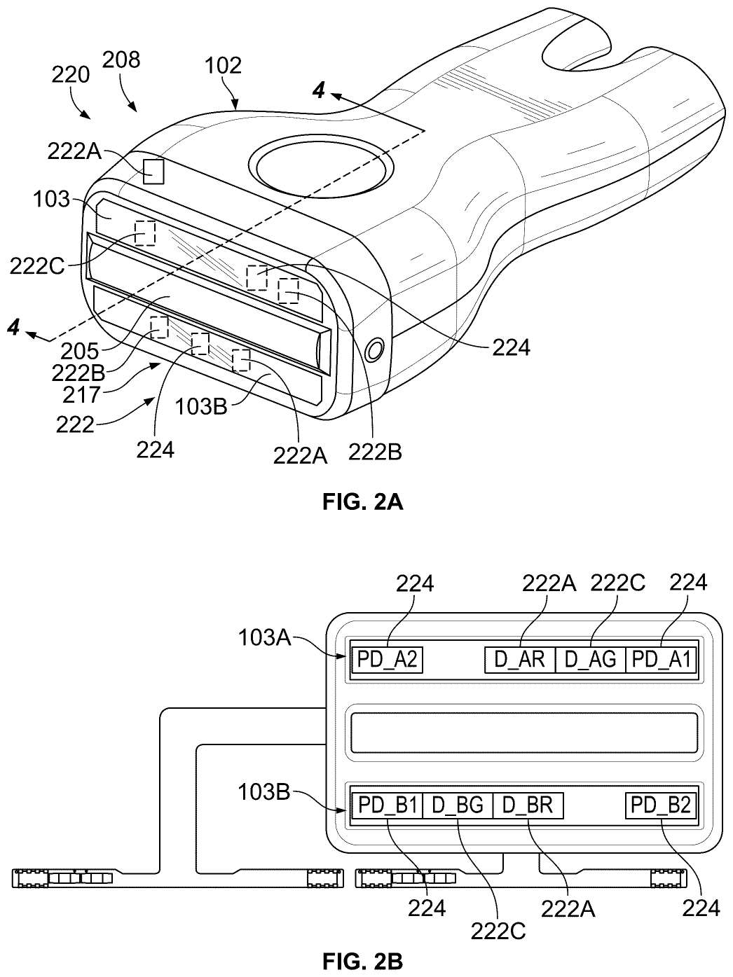

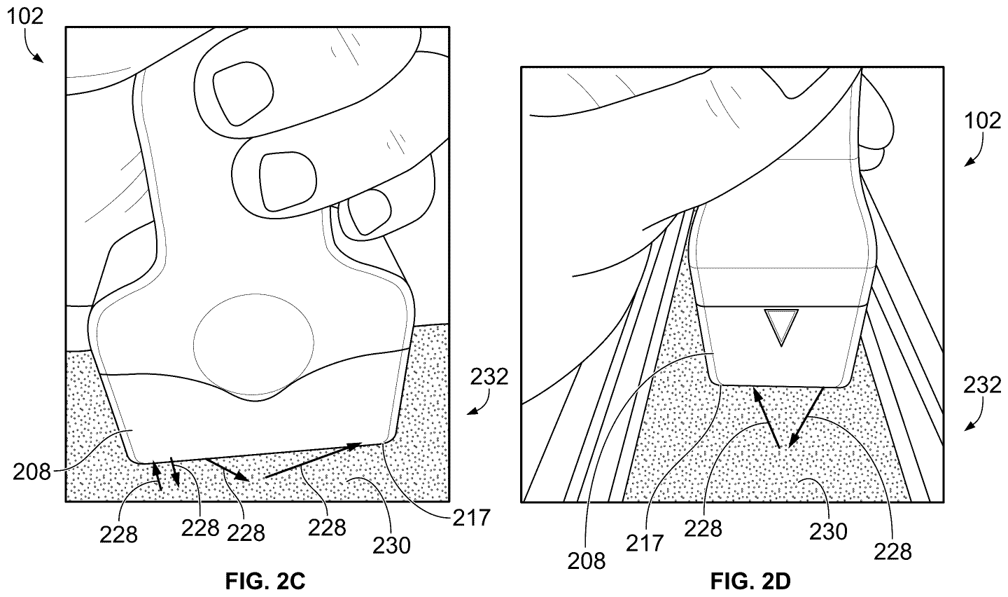

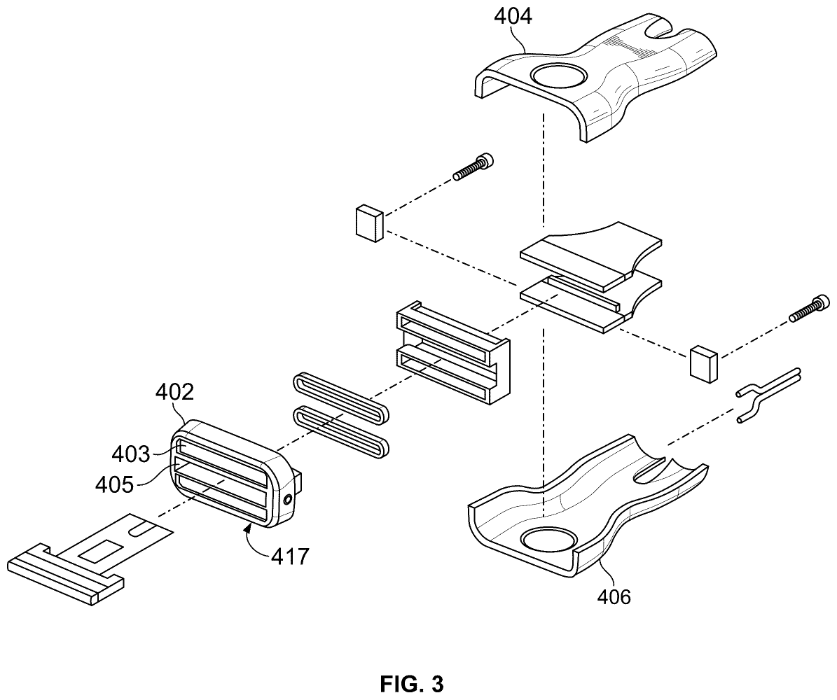

Turning now to FIG. 2A, the optoacoustic probe 102 can also include an ultrasound transducer covered by an acoustic lens 205. The probe 102 includes distal and proximal ends. A probe face 217 of the probe 102 is at the distal end 208. The probe 102 also includes one or more optical windows 103A, 103B through which the light carried along light path 132 can be transmitted to the surface of a volume 160, for example, a three-dimensional volume. The probe 102 may be placed in close proximity with organic tissue, phantom or other volume 160 that may have one or more inhomogeneities 161, 162, such as e.g., a tumor, within. An ultrasound gel (not shown) or other material may be used to improve acoustic coupling between the probe 102 and the surface of the volume 160 and/or to improve optical energy transfer.

At the distal end 208, the probe can include a secondary lighting system 220 that includes one or more auxiliary light sources 222. In one example the one or more auxiliary light sources 222 can be light emitting diodes (LEDs). In example embodiments the LEDs can emit blue light, green light, red light, or a combination thereof. In one example, the one or more auxiliary light sources 222 can be positioned adjacent an optical window 103A, 103B within the housing of the distal end 208. In an embodiment the one or more auxiliary light sources 222 can include directional light that can be directed through an adjacent optical window 103A, 103B to emit light from the auxiliary light source 222 into an environment of the optoacoustic probe 102. In one example each optical window 103A, 103B includes at least one auxiliary light source 222 and the auxiliary light source can be selected based on the light absorption and scattering characteristics of the auxiliary light source in relation to a scanned surface (e.g., the skin of a patient based on skin color).

The secondary lighting system 220 can also include one or more secondary light sensors 224 that are configured to be sensitive to and detect reflected light in the wavelengths of the light emitted by the one or more auxiliary light sources 222. In one example the secondary light sensors 224 can be configured to detect reflected light from an LED. In one example the secondary light sensors 224 can be photodiodes. In another example the secondary light sensors 224 can be positioned within the housing of the optoacoustic probe 102 adjacent to an optical window 103A, 103B, adjacent to the auxiliary light source 222 while not in the direct path of the light emitting from the auxiliary light source 222. The secondary light sensors 224 may be secured on an inner surface of the optoacoustic probe 102, an outer surface of the optoacoustic probe 102, or the like. In one example, secondary light sensors 224 can be located at the edge of the optical windows 103A, 103B. In an example, the secondary light sensors 224 can be positioned to selectively receive scattered light from the probe application area (i.e., adjacent to the probe). In an alternative embodiment, the one or more secondary light sensors 224 are not placed in or adjacent the same optical window 103A, 103B as an auxiliary light source 222. Instead, one or more second light sensors 224 can be positioned adjacent to, or within a different optical window 103A, 103B. In one example the different optical window 103A, 103B may be the optical window adjacent to the ultrasound transducer. The plurality of secondary light sensors 224 may be present to ensure that in certain applications (i.e., safety) adequate contact between the probe face and the diffuse reflective surface of a volume is provided such that the amount of primary light scattered from/through the surface is reduced to a specified limit. When the probe face 217 is in contact with the diffuse reflective surface, the scattering of the light emitted by the auxiliary light sources 222 can be minimized (or no scattering occurs).

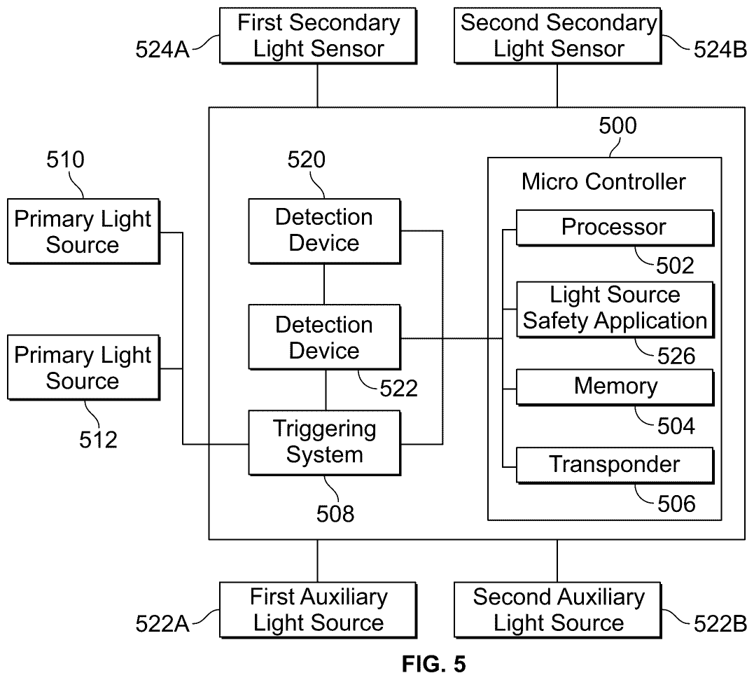

In one embodiment the optoacoustic probe 102 can include a first optical window 103A and second optical window 103B with the ultrasound transducer positioned there between. The auxiliary light sources 222 can include a red-light emitting diode 222A, a blue light emitting diode 222B, and a green light emitting diode 222C. In one example some of the auxiliary light sources 222 can emit the same color wavelength of light, while other auxiliary light sources 222 emit a different color wavelength of light. In one example, a control system (FIG. 5) can be provided that obtains data and information related to the patient, and in one example the skin color of the patient, to determine which light sources, or combination of light sources should be used to detect the skin of the patient. In one example the skin color of the patient may have properties resulting in red light being the most reflective light emitted. In such an example, only an auxiliary light source 222 that emits red light is actuated for determining the location of the imaging area. In another example, green may be the best color for obtaining reflected light from a patient with a different skin color. In yet another example, a color such as orange, purple, yellow, etc. may be the best wavelength of light for obtaining reflections from a skin color. In such example embodiments, a combination of auxiliary light sources 222 can be actuated and light mixed together to form that color desired. In one example the number of auxiliary light sources 222 actuated, intensity of light emitted, or the like may be varied to cause the correct wavelength (e.g., color) and intensity of light from reaching the imaging area. In one example the auxiliary light sources 222 are directional lights such as LEDs that may be positioned to create determined wavelengths of light prior to the determined wavelength of light exiting the interior of the optoacoustic probe via an optical window 103A, 103B.

FIGS. 2B-2D illustrate the same or similar optoacoustic probes 102 having numerous auxiliary light sources 222 in the optical windows 103A, 103B of a probe face 217 as illustrated in FIG. 2A. Added in FIGS. 2C and 2D are light propagation lines 228 that represent the propagation of light from the auxiliary light sources 222 when the probe 102 is in or near contact with a diffuse reflective surface 230 of a volume 232 to be imaged while emitting light from auxiliary light sources 222 in first optical window 103A. FIG. 2C illustrates the light propagation 228 when the probe face 217 is in contact with a diffuse reflective surface 230, while FIG. 2D illustrates the light propagation 228 when the probe face 217 is approaching the diffuse reflective surface 230. In one example the diffuse reflective surface 230 of the volume can include the skin of a patient. In another example the diffuse reflective surface 230 can be the surface of a phantom that represents the volume.

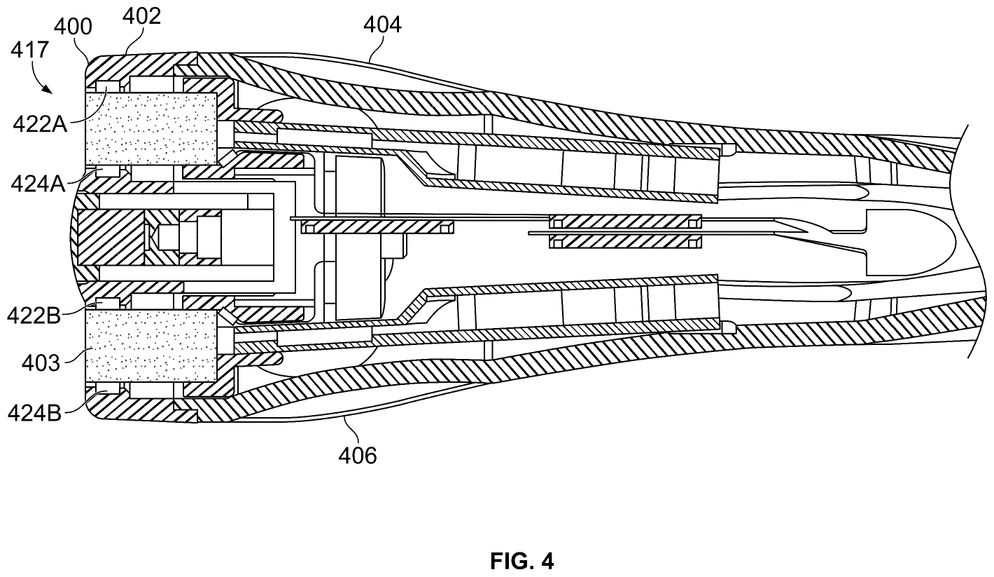

FIGS. 3 and 4 show an exploded view and a cross-sectional view, respectively, of an embodiment of an optoacoustic probe 102 that in one example is the probe 102 shown in FIGS. 1 and 2. In the embodiment of FIGS. 3 and 4, the probe 102 comprises a housing 400 including a distal portion 402 with optical windows 403 and first and second body portions 404, 406 which are shown separated to illustrate the components within the probe 102. The distal portion 402 includes a probe face 417. The distal portion 402 and the first and second body portions 404, 406 may be made from plastic or any other suitable material. Additionally or alternatively, in an embodiment the first and/or second body portions 404, 406 may include one or more regions that are characterized as acoustically reflective, for example, comprising an acoustically reflective material. In this embodiment first and second auxiliary light sources 422A, 422B along with first and second secondary light sensors 424A, 424 are illustrated. The first and second auxiliary light sources 422A, 422B can be of any color of light and be directed to mix the color prior to exiting the optoacoustic probe 102.

FIG. 5 illustrates a block diagram of a computing subsystem, or microcontroller 500 for an optoacoustic probe. In one example the controller and optoacoustic probe are the computing subsystem and optoacoustic probe of FIG. 1. The microcontroller 500 can include one or more processors 502 for making determinations, a memory 504 for storing instructions and historical data, including look up tables, and a transceiver 506 or transponder for receiving and transmitting communication signals to and from the probe 502. In one example the transceiver 506 can be a wireless transceiver that can communicate over a network.

The microcontroller 500 can be coupled to a triggering system 508 for a first light source 510 and a second light source 512. In one example the first light source 510 and second light source 512 can be laser light sources. The triggering system 508 can be configured to independently actuate the first light source 510 and second light source 512. In one example the triggering system can turn off or stop actuating the first light source 510 while continuing to actuate the second light source. Alternatively, the triggering system 508 can simultaneously stop actuation of both the first light source 510 and the second light source 512. In one example the triggering system 508 can be configured to control and vary the amount of voltage input to each of the first light source 510 and second light source 512. In an example, as the voltage input to an individual light source 510, 512 increases, the energy output by that individual light source increases. In one example the increase may be a proportional increase. In one embodiment the triggering system 508 can be configured to increase or decrease the voltage for the first light source 510 while keeping the voltage input to the second light source 512 the same. In one example the triggering system 508 can be configured to increase or decrease the voltage input for the first light source 510 while also increasing or decreasing the voltage input for the second light source. In one example the voltage increase or decrease related to a first light source 510 can be proportional to the voltage increase or decrease related to the second light source 512. Alternatively, the voltage increase or decrease related to the first light 510 source can be disproportional to the increase or decrease related to the second light source 512.

In an example the first and second light sources 510, 512 can emit visible light. In another example the first and second light sources can emit infrared light. In another example the first and third light sources can emit ultraviolet light. In an example the first light source 510 can emit light in a first range of wavelengths while the second light source 512 can emit light in a second range of wavelengths that differs from the first range of wavelengths. In an embodiment the light sources 510, 512 can be a Nd:YAG laser and an Alexandrite laser.

The microcontroller 500 can also include at least one interface. In one example the interface can be a display that can be utilized to display an image by using the optoacoustic probe. The interface can include an input and an output. In one example the interface can be a touch screen that functions as both the input and output. Alternatively, the input can be a keyboard, mouse, microphone, or the like while the output can be a screen, speaker, light indicator, or the like.

The microcontroller 500 can also actuate and operate a first auxiliary light source 522A, a second auxiliary light source 522B, a first secondary light sensor 524A and a second secondary light sensor 524B. While in the example only first and second auxiliary light sources 522A and 522B are illustrated, in other embodiments additional auxiliary light sources may be included. In one example six or more auxiliary light sources may be included. In another example ten or more auxiliary light sources may be provided. In each instance, each auxiliary light source added can provide greater manipulation and functionality for mixing different wavelengths of light together to emit a desired secondary light that is emitted from the optoacoustic probe. In one example, the auxiliary light sources may be replaceable with sockets or ports available to receive the auxiliary light sources along with additional auxiliary light sources. In another example each of the auxiliary light sources 522A, 522B can be on a chip, or printed circuit board that includes light control circuitry for varying characteristics of the light source, including intensity, duration, or the like. In the example, the user can then choose between the functionality of emitting a range of wavelengths and the cost of extra energy and complexity to tune the additional auxiliary light sources.

In one example only one secondary light sensor can be provided, whereas in other examples two, three, five, ten, etc. secondary light sensors may be utilized to increase the amount of information being gathered from the reflected light. In another example the secondary light sensors 524A, 524B may be controlled by a user based on the utility that is needed.

In one embodiment the microcontroller 500 can include a light source safety application 526. The light source safety application 526 can be provided that is configured to control the operation of the auxiliary light sources 522A, 522B. In one example the light source safety application 526 can selectively operate each individual auxiliary light source 522A, 522B independent of the operation of the other auxiliary light sources 522A, 522B. The operation can include the timing of auxiliary light source emission, duration, and intensity. In one example, at the time of the emission of the light source safety application 526 reads, or samples, the value of one or more secondary light sensors 524A, 524B. In one example the sampling may include conditioning of the light sensing hardware to remove noise, increase or decrease the signal value, or the like.

In one example the light source safety application 526 can execute a contact sensing software algorithm which is able to output a value indicating contact or no contact. In an example, the light source safety application 526 can be configured to operate the first and second light sources (e.g., the laser system) 510, 512 to control the output of the first and second light sources 510, 512 based on the contact sensing algorithm to control the output of the first and second light sources 510, 512. In one example the light source safety application 526 can start the primary light emission from a first or second light source 510, 512 or stop/prevent light emission from the first and/or second light source 510, 512. In one example the control system 500 may include both a laser and light sensing circuit that may be controlled by the control system 500.

In one example, the contact sensing algorithm of the light source safety application 526 can be utilized for each optical window and auxiliary light source 522A, 522B. In one example, each auxiliary light source 522A, 522B provides a different output wavelength or color. In one embodiment, a single optical window can be associated with a single auxiliary light source 522A or 522B, and/or numerous auxiliary light sources 522A or 522B. In one example one auxiliary light source 522A or 522B can be disposed in the optical window and another auxiliary light source 522A or 522B can be coupled outside of the optical window.

In an example, for the contacting sensing algorithm to determine contact of a probe face to the skin, total probe face surface contact is required and only a single wavelength or color is utilized. In other examples, one or more optical windows can be utilized and multiple different auxiliary light sources of different wavelengths, or color are used. In an example, the output of the contact sensing algorithm from each optical window and auxiliary light source color may be combined with another auxiliary light source color to form a combined contact sensing output. In another example, a combined contact sensing algorithm is applied to determine overall contact status.

In one example the light source safety application 526 can pulse an auxiliary light source output at the same time the readings from the secondary light sensors 524A, 524B are sampled. For example, in an embodiment where a first auxiliary light source 522A is red, the first auxiliary light source 522A can pulse Red LED D_AR in optical window A and readings from all of the secondary light sensors 524A, 524B are collected (i.e., PD_A1, PD_A2, PD_B1, PD_B2). For each reading collected the following algorithm can be applied:
Compare the output of each secondary light sensor 524A, 524B (e.g., photodiode) to a threshold:
Contact_PD_A1=PD_A1>CONTACT_THRESHOLD_NEAR
Contact_PD_A2=PD_A2>CONTACT_THRESHOLD_NEAR
Contact_PD_B1=PD_B1<CONTACT_THRESHOLD_FAR
Contact_PD_B2=PD_B2<CONTACT_THRESHOLD_FAR
Window_A_Contact_Red=Contact_PD_A1 AND Contact_PD_A2 AND Contact_PD_B1 and Contact_PD_B2

In one example the Window_A_Contact_Red threshold may be modified to be a majority vote system whereas a plurality of secondary light sensor readings determines the contact status. In this case 3 out of 4 may be applied. In one example the following algorithm may be used in practice to account for manufacturing variations or erroneous readings.

$$Window\_A \_Count \_Re d = count ( Contact\_PD \_… ) > 3$$

Selection of CONTACT_THRESHOLD_NEAR and CONTACT_THRESHOLD_FAR are optimized to allow operation over a wide range of tissue characteristics (i.e., skin tone and skin reflectivity) while also accounting for imperfect contact with tissue due to anatomical feature such as nipples, scars, or curvatures. The thresholds are specific to a given light source color. CONTACT_THRESHOLD_NEAR determines the minimum amount of light allowed for a detection to occur; and thus, results in how sensitive the probe is to the tissue and skin color of a patient. CONTACT_THRESHOLD_FAR determines the maximum amount of light for allowed for a detection to occur; and thus, results how far the probe can be held from the surface.

In one example the light source safety application 526 determines the operation of the auxiliary light sources 522A, 522B based on the skin color of a patient. In one example the Fitzpatrick Type I to Type VI skin color is utilized, while in other examples the Von Luschan scale can be utilized. In each instance, the scales provide guidance to how different wavelength light is diffused and/or reflected off the skin of a patient based on the tone or skin color of the patient. Each scale can be approximated to a red, green, blue color scale that is typical of LED light sources. By using the scales, the determinations can be made related to the skin color of a patient to confirm an identified skin color. In one example the skin color of a patient can be input by a clinician and then confirmed based on characteristics of interest of signals received by the secondary light sensors 524A, 524B. In the color input, based on the characteristics of interest such as percentage of reflective light captured, absorption percentage, or the like, does not match with expected values for the light being emitted by the auxiliary light sources 522A, 522B based on skin color, the light source safety application 526 can vary inputs to the auxiliary light sources 522A, 522B to provide a better output for the skin color of the patient. Alternatively, instead of varying inputs of the auxiliary light sources, in one example settings or parameters (e.g., gain) of at least one of the secondary light sensors can be varied to provide sufficient contrast between surface contact and non-surface contact. In one example, to accomplish, a calibration process can be provided where when an optoacoustic prove contacts a surface of a volume, an integration window can be adjusted to a maximum signal, whereas when the probe is in free air the secondary light sensors can be offset to nearly zero. Once initial tuning of gain and offsets are completed, additional calibration may be performed to account for probe variations.

In another example, the light source safety application 526 can be configured to tune either the auxiliary light sources 522A, 522B or secondary light sensors 524A, 524B. In one example tuning can be provided to ensure the most absorptive/least scattering surface can still be detective. In one example, the probe can be positioned above a target surface of a volume at a determined contact distance. At this time readings of the secondary light sensors 524A, 524B can be obtained. Based on the obtained readings, a contact threshold near value that is lower than the average reading can be selected. In one example, this procedure can be repeated for numerous different surfaces having different characteristics such as color, consistency, and the like. For example, for a system with one auxiliary light source wavelength/color, a contact threshold can be selected to allow the most absorptive surface to activate the contact sensing while preventing emission from a large distance/reflective surface. For a system with more than one auxiliary light wavelength/color, multiple threshold levels may be used. In addition to this, a second threshold, contact threshold near, can be used to prevent activation with light scatters off the surface into secondary light sensors 524A, 524B, outside of the emitting auxiliary light source optical window.

In yet another example, the light source safety application 526 can also be configured to tune for medium thresholds in addition to the contact threshold near and far. In example embodiments a when a probe is positioned slightly above a diffuse surface (e.g., reflective target) of a volume, light scatters from the emitting auxiliary light source 522A, 522B in an optical window into the adjacent secondary light sensor 524A, 524B. Light also scatters off the surface of the surface (e.g., reflective target) and into the auxiliary light sources in the adjacent optical windows (PD_B1 and PD_B2). In the example, more intense light is expected in PD_B2. The amount of scattered light can be used to determine as a secondary check for contact with a reflective surface and is denoted as CONTACT_THRESHOLD_FAR. Ideally with perfect contact with tissue, the amount of light emitting from optical window should be 0 in PD_B1 and PD_B2; however in application, where surfaces are not ideally flat (i.e., breast tissue around the nipple, we need to account for stray reflections); thus the threshold is non-zero.

In the example, when determining CONTACT_THRESHOLD_FAR experimental tuning of the threshold is selected to ensure that the most absorptive/least scattering surface can be detective. The probe is positioned above the target surface at a desired contact distance. Readings are then obtained from the one or more secondary light sensors 524A, 524B, and a CONTACT_THRESHOLD_FAR value is selected that is higher than the average reading. This process can then be repeated for several different surfaces. For a system with one auxiliary light source wavelength/color, the threshold needs to be selected to allow the most absorptive surface to activate the contact sensing while preventing emission from a large distance/reflective surface. For probes with auxiliary light sources having more than one wavelength/color; multiple threshold levels may be used. Both values of CONTACT_THRESHOLD_NEAR and CONTACT_THRESHOLD_FAR can be individually tuned accordingly. The outputs from each individual optical window/auxiliary light source wavelength/color may be combined to make an overall combined contact decision. In one implementation, one may require all of the following conditions to be true:
Overall_Contact=Window_A_Contact_Red AND Window_B_Contact_Red etc. for a probe having two windows A and B and a single LED Red.
Or one may target a plurality of skin tones or tissues with multiple auxiliary light sources:
Overall_Contact=(Window_A_Contact_Red AND Window_B_Contact_Red) OR (Window_A_Contact_Green AND Window_B_Contact_Green)

This concept can be extended to a plurality of windows 103A, 103B. If there are more than three windows, then:
Overall_Contact=count(window_contact)>2

In the ideal case when extremely far from any reflective surface of a volume, no light should be detected from the secondary light sensors 524A, 524B within the same optical window by the secondary light sensor 524A, 524B furthest from the auxiliary light source 522A or 522B. Some stray light may be detected in the secondary light sensor 524A or 524B closest to the auxiliary light source 522A or 522B. In this moment—the readings from all secondary light sensors should be zero. This case rarely occurs due to manufacturing variances in assembly and parts. Initially an offset is determined using the contact algorithm while in this state (with no reflective surface); however periodically a self calibration routine may be needed to adjust the offset.

During manufacturing, variations of placement of the auxiliary light sources 522A, 522B and secondary light sensors 524A, 524B can occur. The variation can result in different threshold values batch to batch. To address, the threshold determined can be selected based on a sampling plan. Movement of auxiliary light sources 522A, 522B and secondary light sensors 524A, 524B over time can also result in different threshold values batch to batch. To address, the contact algorithm accounts for these variations. In other embodiment the auxiliary light sources and/or secondary light sensors may fail over time. By having redundant auxiliary light sources or secondary light sensors and using the contact algorithm can account for failures by discarding inputs from bad auxiliary light sources and/or secondary light sensors. To account for the variations the self calibrating contact algorithm may be applied to adjust the offsets in the secondary light sensors 524A, 524B.

The light source safety application 526 can adjust the reading of each secondary light sensor 524A, 524B to account for sources of noise, or internal scattering from non-ideal placement of the auxiliary light sources 522A, 522B and the secondary light sensors 524A, 524B. One example, the auxiliary light sources 522A, 522B and secondary light sensors 524A, 524B within the same optical window can be positioned such that light from the emitting auxiliary light source 522A or 522B in the same optical window does not reach the sensing auxiliary light source in the same optical window. This situation can occur when the secondary light sensor 524A or 524B is placed next to the emitting auxiliary light source 522A or 522B, or the emitting auxiliary light source 522A, 522B can be angled within the optical window such that internal scattering of light reaches the secondary light sensors 524A, 524B. In a condition where the emitting auxiliary light sources' light is away from contact with a reflective surface (i.e., emitting in air) the secondary light sensor readings from the sensing auxiliary light sources are sampled. A plurality of readings can be taken from each secondary light sensors and then averaged. The averaged value can be identified as the offset. An offset can be computed, determined, calculated, etc. for each secondary light sensor and then subtracted from the secondary light sensor reading when not in the calibration state.

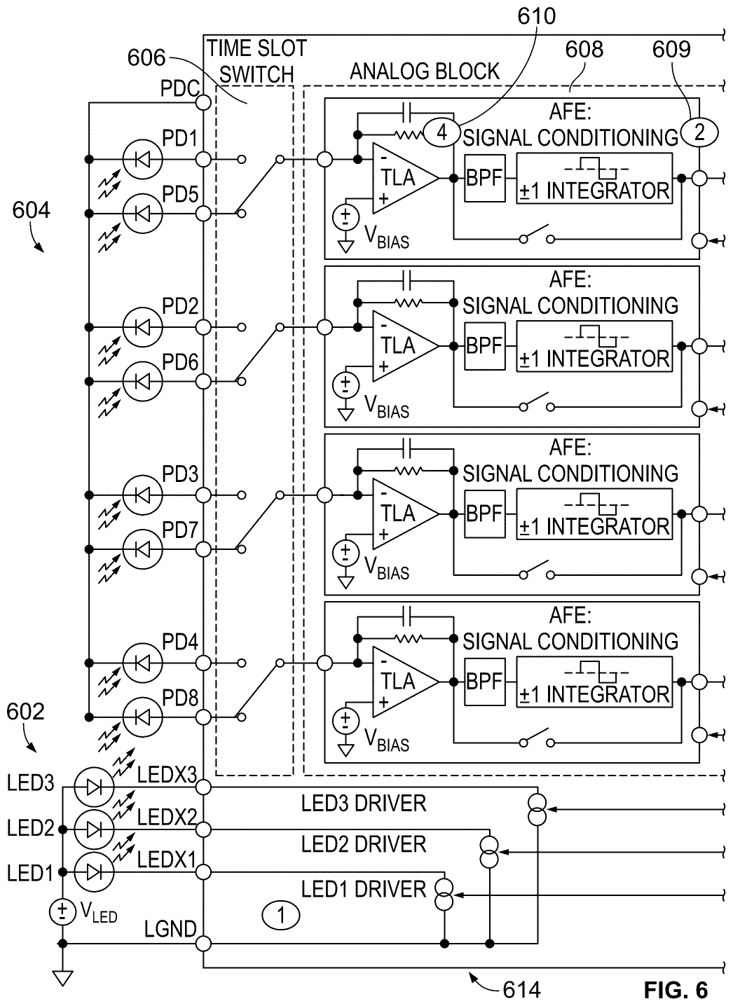

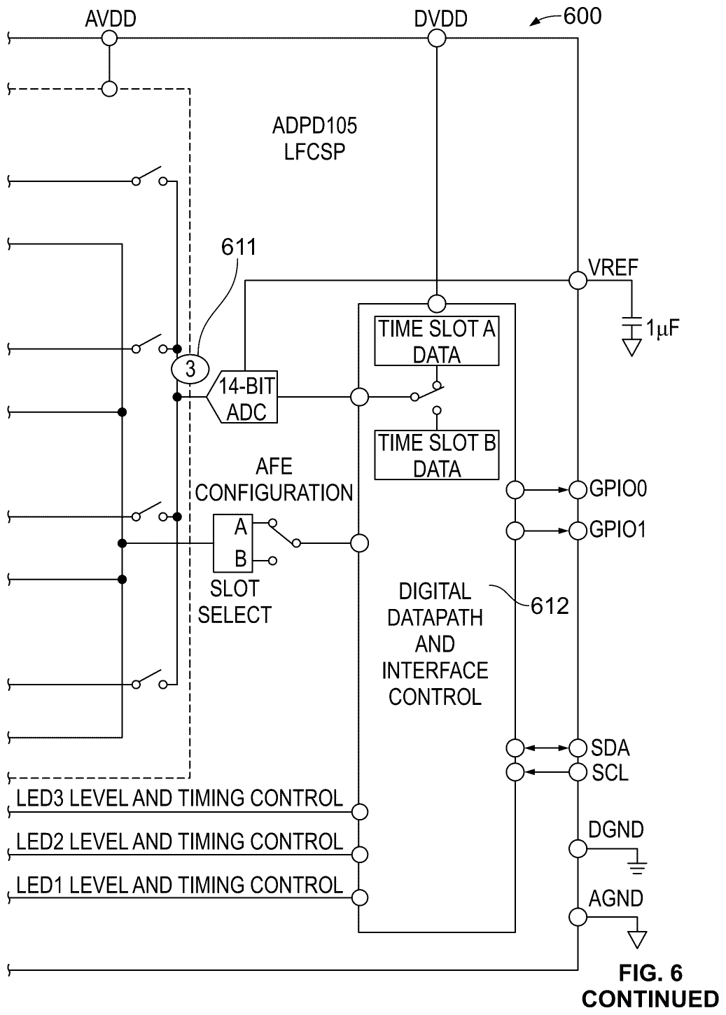

FIG. 6 illustrates example light control circuitry 600 configured to tune the output of auxiliary light sources 602 based on inputs received from secondary light sensors 604. As illustrated, a time slot switch 606 can be provided to vary the source of data (e.g., the secondary light sensor 604) from which signals are received. An analog block 608 then receives the inputs based on the time slot switch 606 configuration and can be configured to condition the signals received from the secondary light sensors 604. For examples, the analog block can include first components 609 to adjust or vary an integration window and/or second components 610 to adjust or vary the gain of the signal. The analog block can also be configured to tune analog to digital current (ADC) offsets 611 prior to the signals reaching a digital datapath and interface control 612 that can be configured to provide control signals for the auxiliary light sources 602 based on the signals obtained by the secondary light sensors 604. In one example the control signals include timing signals along with intensity level signals that can be provided to drivers 614 of the auxiliary light sources 602. In one example the drivers 614 can be LED drivers when the auxiliary light sources 602 are LED light sources. In one example the drivers are configured to determine the current source to the auxiliary light sources 602 to provide an input for each auxiliary light source 602. In one example, the determination can be based on the detected skin color of the patient, the distance detected between a patient and an optoacoustic probe, or the like.

Appendix A as attached hereto presents the computer based code for threshold detection that can be utilized to configure the light control circuitry 600 of FIG. 6. This code can be used by any software associated with the light control circuitry, including the interface control 612 that causes the functionality of the light control circuitry 600.

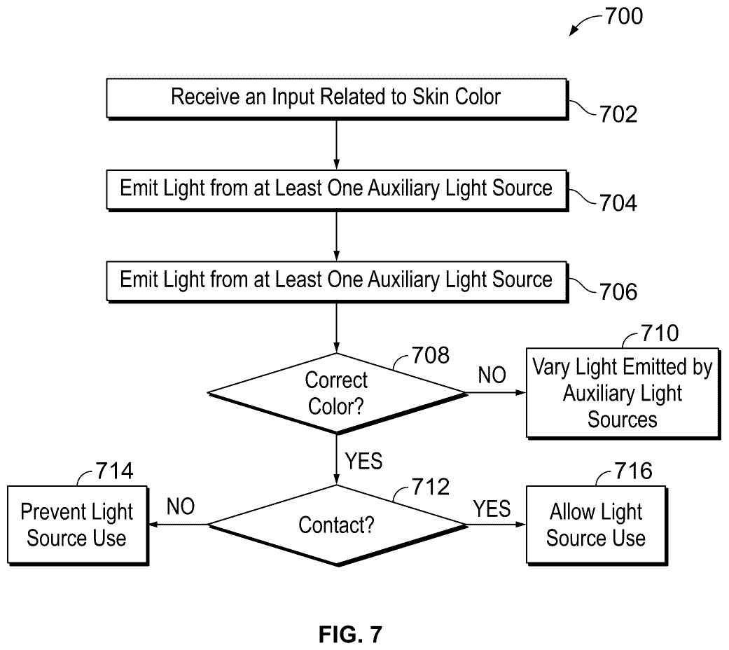

FIG. 7 illustrates a method 700 of controlling light sources of an optoacoustic probe. In example embodiments the optoacoustic systems, optoacoustic probes, controllers, etc. previously described in FIGS. 1-6 are used to complete one or more steps of the method 700.

At 702, one or more processors receive an input related to a skin color of a patient. In one example the skin color can be based on the Fitzpatrick Type I to Type VI skin color, while in other examples the Von Luschan scale can be utilized. A user can input the skin color into the one or more processors to provide a baseline skin color of the patient.

At 704, light is emitted from at least one auxiliary light source based on the skin color input by the user as an optoacoustic probe moves towards a surface of a volume to be scanned. In one example the auxiliary light source can be an LED light source that is within an optical window of the distal end of the probe. The auxiliary light source can emit a determined wavelength or color of light based on the input skin color.

At 706, at least one secondary light sensor detects and/or samples the reflection of the light emitted by the at least one auxiliary light source off of the surface to be exampled. In one example, the secondary light sensor can be a pyroelectric sensor, a photodiode sensor, or the like.

At 708, a determination is made whether the input skin color is a correct skin color based on the reflected light sensed by the at least one secondary light sensor. If the input skin color is incorrect, then at 710, the one or more processors varies the light emitted by the at least one auxiliary light sources. In one example, the selection of auxiliary light sources can be varied to present different wavelengths or colors of light. In other examples, the intensity of one or more auxiliary light sources can be varied.

If the skin color is determined to be correct, or after adjustment of the light emitted by the auxiliary light sources, at 712, a determination is made whether the probe face is contacting the surface to be examined. In one example a contact algorithm may be utilized based on characteristics of interest (COIs) of the auxiliary light sources. The COIs can include light wavelength, light intensity, or the like.

If at 712 a determination is made that the probe face is not contacting the surface to be examined, then at 714 the one or more processors prevent light sources of the optoacoustic probe system from emitting light. In one example the light sources are laser light sources, and even if a triggering system is actuated by a user, light will not be emitted via the probe. Alternatively, if a determination is made that the probe face is contacting the surface to be examined, then at 716 the light sources are permitted to emit light onto the surface.

The present system and methods are described above with reference to block diagrams and operational illustrations of methods and devices comprising an optoacoustic probe. It is understood that each block of the block diagrams or operational illustrations, and combinations of blocks in the block diagrams or operational illustrations, may be implemented by means of analog or digital hardware and computer program instructions. These computer program instructions may be provided to a processor of a general purpose computer, special purpose computer, ASIC, FPGA or other programmable data processing apparatus, such that the instructions, which execute via the processor of the computer or other programmable data processing apparatus, implements the functions/acts specified in the block diagrams or operational block or blocks. In some alternate implementations, the functions/acts noted in the blocks may occur out of the order noted in the operational illustrations. For example, two blocks shown in succession may in fact be executed substantially concurrently or the blocks may sometimes be executed in the reverse order, depending upon the functionality/acts involved.

As used in this description and in the following claims, “a” or “an” means “at least one” or “one or more” unless otherwise indicated. In addition, the singular forms “a”, “an”, and “the” include plural referents unless the content clearly dictates otherwise. Thus, for example, reference to a composition containing “a compound” includes a mixture of two or more compounds.

As used in this specification and the appended claims, the term “or” is generally employed in its sense including “and/or” unless the content clearly dictates otherwise.

The recitation herein of numerical ranges by endpoints includes all numbers subsumed within that range (e.g., 1 to 5 includes 1, 1.5, 2, 2.75, 3, 3.80, 4, and 5).

Unless otherwise indicated, all numbers expressing quantities of ingredients, measurement of properties and so forth used in the specification and claims are to be understood as being modified in all instances by the term “about,” unless the context clearly dictates otherwise. Accordingly, unless indicated to the contrary, the numerical parameters set forth in the foregoing specification and attached claims are approximations that can vary depending upon the desired properties sought to be obtained by those skilled in the art utilizing the teachings of the present invention. At the very least, and not as an attempt to limit the scope of the claims, each numerical parameter should at least be construed in light of the number of reported significant digits and by applying ordinary rounding techniques. Any numerical value, however, inherently contains certain errors necessarily resulting from the standard deviations found in their respective testing measurements.

Those skilled in the art will recognize that the methods and systems of the present disclosure may be implemented in many manners and as such are not to be limited by the foregoing example embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible. Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

Various modifications and alterations to the invention will become apparent to those skilled in the art without departing from the scope and spirit of this invention. It should be understood that the invention is not intended to be unduly limited by the specific embodiments and examples set forth herein, and that such embodiments and examples are presented merely to illustrate the invention, with the scope of the invention intended to be limited only by the claims attached hereto. Thus, while the invention has been particularly shown and described with reference to a preferred embodiment thereof, it will be understood by those skilled in the art that various changes in form and details may be made therein without departing from the spirit and scope of the invention.

What is claimed is:

1. An optoacoustic probe for optoacoustic imaging of a volume, the optoacoustic probe having a distal end operable to contact the volume and a proximal end, the optoacoustic probe comprising:
a light source outside a probe housing configured to generate light that is transmitted along a light path to generate optoacoustic return signals when the light reacts with the volume;
an optical window configured to carry the light along the light path to the volume;
an auxiliary light source coupled to the probe housing and configured to generate auxiliary light carried through the optical window to the volume during operation of the optoacoustic probe;
a secondary light sensor configured to detect signals generated from the auxiliary light source reflecting from the volume; and
a microcontroller including one or more processors, and a memory coupled to the one or more processors, wherein the memory stores program instructions, wherein the program instructions are executable by the one or more processors to:
receive the signals generated from the auxiliary light reflecting from the volume;
determine contact between the volume and the optoacoustic probe based on the signals generated from the auxiliary light reflecting from the volume and color of a surface of the volume; and
prevent the light from the light source emitting from the probe housing until the optoacoustic probe is contacting the volume.

2. The optoacoustic probe of claim 1, wherein the one or more processors include program instructions to vary an output of the auxiliary light source based on the color of the surface of the volume.

3. The optoacoustic probe of claim 1 wherein the auxiliary light source is a first auxiliary light source configured to emit a first wavelength of light, and the optoacoustic probe further comprises a second auxiliary light source configured to emit a second wavelength of light.

4. The optoacoustic probe of claim 3, wherein the first wavelength of light and the second wavelength of light are different wavelengths.

5. The optoacoustic probe of claim 3, wherein the one or more processors include program instructions to select between the first auxiliary light source and the second auxiliary light source based on the color of the surface of the volume.

6. The optoacoustic probe of claim 3, wherein the one or more processors include program instructions operate the first auxiliary light source and second auxiliary light source to mix the first wavelength of light with the second wavelength of light based on the color of the surface of volume.

7. The optoacoustic probe of claim 1, wherein the light source is a laser light source and the auxiliary light source is a light emitting diode.

8. The optoacoustic probe of claim 1, wherein the secondary light sensor is a photodiode.

9. The optoacoustic probe of claim 1, further comprising a triggering system configured to actuate the light source and the auxiliary light source and configured to prevent actuation of the light source before actuation of the auxiliary light source.

10. The optoacoustic probe of claim 1, wherein the auxiliary light source is disposed adjacent the optical window at the distal end of the optoacoustic probe.

11. The optoacoustic probe of claim 1, wherein the one or more processors are configured to receive the signals generated from the auxiliary light reflecting from the volume while the light source generates the light.

12. A method of triggering a light source of an optoacoustic probe comprising:
selecting an auxiliary light source from plural auxiliary light sources based on a color of a surface of a volume;
operating the auxiliary light source selected to generate an auxiliary light carried through an optical window to the volume during operation of the optoacoustic probe;
detecting the auxiliary light after the auxiliary light has reflected from the surface of the volume;
determining when the optoacoustic probe contacts the volume based on the auxiliary light after the auxiliary light has reflected from the volume;
triggering the light source in response to determining the optoacoustic probe contacts the volume; and
preventing the light source from actuating in response to determining the optoacoustic probe is not contacting the volume.

13. The method of claim 12, further comprising varying an output of the auxiliary light source based on the color of the surface of the volume.

14. The method of claim 12, wherein selecting the auxiliary light source from the plural auxiliary light sources includes identifying a first auxiliary light source configured to emit a first wavelength of light; identifying a second auxiliary light source configured to emit a second wavelength of light; and selecting the first auxiliary light source as the auxiliary light source based on the color of the surface of the volume.

15. The method of claim 12, wherein the auxiliary light source is a first auxiliary light source, and the method further comprises mixing a first wavelength of light emitted by the first auxiliary light source with a second wavelength of light emitted by a second auxiliary light source based on the color of the surface of the volume.

16. The method of claim 12, further comprising varying an intensity of the auxiliary light source based on the color of the surface of the volume.

17. The method of claim 12, further comprising preventing actuation of the light source before actuation of the auxiliary light source.

18. An optoacoustic system for optoacoustic imaging of a volume, comprising:
an optoacoustic probe having a distal end operable to contact a surface of the volume;
a laser light source in a chassis coupled to the optoacoustic probe via an optoacoustic cable, the laser light source configured to generate light that is transmitted along a light path within the optoacoustic cable to generate optoacoustic return signals when the light reacts with the volume;
an optical window configured to carry the light along the light path to the volume;
a first auxiliary light source positioned adjacent to the optical window at the distal end of the optoacoustic probe and configured to emit a first wavelength of light;
a second auxiliary light source posited adjacent to the optical window at the distal end of the optoacoustic probe and configured to emit a second wavelength of light;
a microcontroller including one or more processors, and a memory coupled to the one or more processors, wherein the memory stores program instructions, wherein the program instructions are executable by the one or more processors to:
select at least one of the first auxiliary light source and the second auxiliary light source based on a color of the surface of the volume;
operate auxiliary light source selected; and
prevent the light from the laser light source from emitting based on signals generated from the auxiliary light source selected.

19. The optoacoustic system of claim 18, further comprising secondary light sensors configured to receive the signals generated from the auxiliary light source selected.

20. The optoacoustic system of claim 19, wherein the one or more processors include program instructions to vary an output of the auxiliary light source selected based on the color of the surface of the volume.
